(19) United States
(12) Patent Application Publication
(10) Pub. No.: US 2018/0153734 A1
(43) Pub. Date: Jun. 7, 2018

(54) IMPLANT AND IMPLANTATION TOOL ADAPTED FOR OCCLUDING FALLOPIAN TUBES OF PLACENTAL MAMMALS

(71) Applicant: The Trustees of Dartmouth College, Hanover, NH (US)

(72) Inventors: Ulrike G.K. Wegst, Hanover, NH (US); Prajan Divakar, Hanover, NH (US); B. Stuart Trembly, Hanover, NH (US); P. Jack Hoopes, Hanover, NH (US); James D. Petryk, White Bear Lake, MN (US); Daniel Santana, Brookhaven, NY (US)

(21) Appl. No.: 15/833,976

(22) Filed: Dec. 6, 2017

Related U.S. Application Data
(60) Provisional application No. 62/430,850, filed on Dec. 6, 2016.

(51) Int. Cl. A61F 6/22; A61L 31/04; A61L 31/14; A61L 31/02; A61B 18/04; A61B 17/32; A61B 18/14; A61B 18/06

(52) U.S. Cl. CPC A61F 6/225; A61L 31/044; A61L 31/146; A61L 31/022; A61L 31/148; A61B 2018/00595; A61B 18/04; A61B 17/32; A61B 18/1485; A61B 18/06; A61L 31/042

(57) ABSTRACT

An implantable radially-porous, polymeric, scaffold device configured for insertion into and obstruct a Fallopian tube formed of a polymer such as collagen, nanocellulose, xanthan gum, konjac glucomannan, alginate, agar, agarose, chitin, chitosan, chitosan-alginate polylactic acid (PLA), polygultamic acid (PGA), polycaprolactone, or polydioxanone. The device is compressed and placed within a first end of an insertion tube adapted for insertion through the cervix into the Fallopian tube; the insertion tube having a plunger to expel the device from the tube. The method includes using the insertion tube containing the radially freeze-cast and freeze-dried implant. Under hysteroscopic guidance the tube is positioned in the Fallopian tube and the implant is expelled into the Fallopian tube. Epithelium of the Fallopian tube is disrupted; and ingrowth of cells into the implant is permitted.

### CROSS-REFERENCE TO RELATED APPLICATIONS

The present application claims the benefit of and priority to U.S. Provisional Patent Application 62/430,850 filed Dec. 6, 2016, the entire contents of which are incorporated herein by reference.

### GOVERNMENT INTEREST

This invention was made with government support under Grant No. R21 HD 087828.01 awarded by the National Institutes of Health. The government has certain rights in the invention.

### BACKGROUND

Female placental mammals, including species such as dogs, cats, humans, cattle, and horses, typically release an oocyte from an ovary into the abdominal cavity. This oocyte is collected by fimbriae, projections of fallopian tubes that surround the ovary, and swept into a lumen of the fallopian tube. The fallopian tube provides an environment in which the oocyte may be fertilized by sperm to form a zygote while the fallopian tube conducts the oocyte or zygote into the uterus. Zygotes develop into blastocysts that may then implant into uterine lining, where the blastocyst forms an infant of the species, with placenta, and associated membranes.

Female sterilization is a popular form of birth control for humans worldwide; being the most used form of contraception in the US, India, China, and some other populous countries. Sterilization is typically accomplished by obstructing the fallopian tubes to prevent oocytes from being fertilized into zygotes, unfertilized oocytes trapped in fallopian tubes then degrade and are absorbed. Obstruction has been performed by surgically cutting and tying off the fallopian tubes. Obstruction has also been accomplished by inserting, via the transvaginal-transcervical-transuterine (TV-TC-TU) route, a nickel-titanium obstructive device insert such as Essure, (trademark of Bayer Healthcare Pharmaceuticals), or inserting along the TV-TC-TU route an injector through which silicone is inserted to form in place a silicone plug as in the Ovabloc intratubal device withdrawn from the marketplace in 2009.

FDA investigation of Essure suggests there is potential market for an alternative nonsurgical, non-hormonal TV-TC-TU device that can be implanted without anesthesia and without requiring complex and expensive imaging technologies such as X-ray, computed tomography (CT), or magnetic resonance imaging (MRI).

### SUMMARY

In an embodiment, a radially-porous polymeric scaffold implantable device configured for insertion into a Fallopian to obstruct the fallopian tube, the device comprising a polymer such as collagen, cellulose, chitin, chitosan, alginate, agar, agarose, gelatin, soy protein, hyaluronic acid, elastin, silk, xanthan gum, konjac glucomannan, polylactic acid (PLA), polygultamic acid (PGA), polycaprolactone, or polydioxanone, or any combination thereof. The device is compressed and placed within the tip of an insertion tube adapted for insertion through the vagina, cervix, and uterus into the Fallopian tube; the insertion tube having a plunger to expel the device from the tube.

In another embodiment, a method of sterilizing a female placental mammal includes using the insertion tube containing a radially freeze-cast and freeze-dried implant comprising collagen, cellulose, chitin, chitosan, alginate, agar, agarose, gelatin, soy protein, hyaluronic acid, elastin, silk, xanthan gum, knock galactomannan, polylactic acid (PLA), polygultamic acid (PGA), polycaprolactone, or polydioxanone, or a combination thereof. Prior to implanting the device, the epithelium of the Fallopian tube is disrupted mechanically. Under hysteroscopic guidance the tube is positioned in the Fallopian tube and the implant is expelled into the Fallopian tube; and ingrowth of cells into the implant is permitted.

In another embodiment, a method of sterilizing a female placental mammal includes using the insertion tube containing a radially freeze-cast and freeze-dried implant comprising magnetic nanoparticles and a polymer comprising collagen, cellulose, chitin, chitosan, alginate, agar, agarose, gelatin, soy protein, hyaluronic acid, elastin, silk, xanthan gum, konjac glucomannan, polylactic acid (PLA), polygultamic acid (PGA), polycaprolactone, or polydioxanone, or a combination thereof. Prior to implanting of the device, the epithelium of the Fallopian tube is disrupted using heat. Under hysteroscopic guidance the tube is positioned in the Fallopian tube and the implant is expelled into the Fallopian tube. Ingrowth of cells into the implant is permitted.

In another embodiment, a method of sterilizing a female placental mammal includes using the insertion tube containing a radially freeze-cast and freeze-dried implant comprising a biopolymer or biodegradable polymer. In particular embodiments the biopolymer or biodegradable polymer is selected from collagen, nanocellulose, xanthan gum, konjac glucomannan, alginate, agar, agarose, chitin, chitosan, chitosan-alginate polylactic acid (PLA), polygultamic acid (PGA,) polycaprolactone, or polydioxanone. Under hysteroscopic guidance the tube is positioned in the Fallopian tube and the implant is expelled into the Fallopian tube. Post implantation, the epithelium of the Fallopian tube is disrupted by either applying an AC magnetic field to magnetic nanoparticles embedded within the implant or infiltrating the implant with a strong chemical irritant; and ingrowth of cells into the implant is permitted.

### BRIEF DESCRIPTION OF THE FIGURES

FIG. 1A and FIG. 1B are cross sections of an implant.

FIG. 1C is a cross section of a radially-porous implant formed from crosslinked bovine collagen.

FIG. 1D is a section of a longitudinally-porous implant formed from crosslinked collagen showing longitudinal pores.

FIG. 1E is a section of a radially porous implant formed from crosslinked collagen showing pore structure.

FIG. 2 is a photograph of a model showing an implant within a Fallopian tube.

FIG. 3 is a flowchart of a method of sterilizing female placental mammals.

FIG. 4A and FIG. 4B illustrate devices for mechanical de-epithelialization of Fallopian tube lining prior to inserting the radially-porous implant.

FIG. 5 is a micrograph showing cellular infiltration into a crosslinked 2% bovine collagen scaffold of 2 mm uncompressed diameter 6 days after implantation into a rat uterine horn.

FIG. 6 is a micrograph showing a rat uterine horn having lumen obliterated following mechanical disruption of tube lining and insertion of a crosslinked 2% bovine collagen scaffold of 2 mm uncompressed diameter.

### DETAILED DESCRIPTION OF THE EMBODIMENTS

We propose a three-dimensional, elongated, porous, implantable sterilization device for occluding the Fallopian tubes, where the device is formed of material that allows ingrowth of cells from the tube lining, and the nonmetallic material of the implant biodegrades over time.

The implantable device includes a porous polymeric material in the shape of a cylinder. The device is placed inside a patient's fallopian tubes, in various embodiments with or without prior de-epithelialization to cause cell growth, after which cells grow into the device and blocking the tubes. After implantation, the device naturally degrades and the patient is left with a blocked fallopian tube.

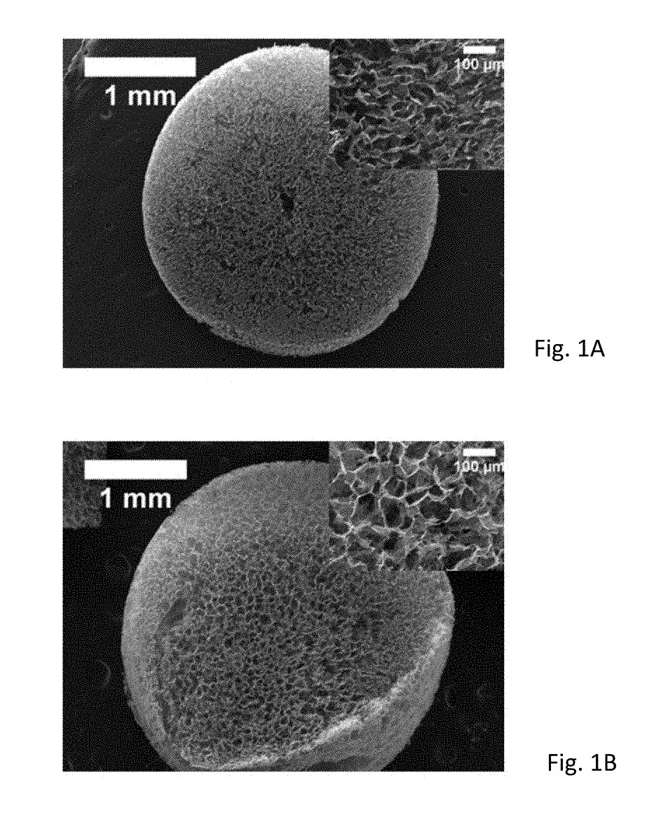

For purposes of this document, a scaffold is a three-dimensional shape formed of a low-to-mid density porous material having interconnecting pores like an open-celled foam. A directionally-porous scaffold has oblong or elongated pores, where a majority of pores in each part of the scaffold measure longer in a particular pore-axis direction than in directions perpendicular to that pore-axis direction, and where the pore-axis directions approximately align with a preferred direction; the scaffold is then directionally-porous along that preferred direction. A radially-porous scaffold is a directionally-porous scaffold where the preferred pore-axis direction aligns with radii from a central axis of the scaffold towards a perimeter of the scaffold. We have demonstrated formation of radially-porous scaffolds having microstructure with a majority of pores elongated along radii from a central axis of the scaffold towards a scaffold perimeter, as seen most easily in FIG. 1C. We have also demonstrated fabrication of longitudinally-porous scaffolds as illustrated in FIG. 1D, showing a preferred pore direction aligned along a central axis of the implant. We have also demonstrated fabrication of porous scaffolds having mixed microstructure.

We achieve radially-porous scaffolds by radial freeze-casting in a high-thermal-conductivity mold that encourages ice-crystal formation inwards towards an implant axis from mold periphery; the mold is placed on a cold finger of the freeze-caster. We achieve longitudinally-porous scaffolds by freeze-casting using a high-thermal-conductivity cap at one end of a lower-thermal-conductivity mold. The cap is then placed on a cold finger of the freeze-caster.

The scaffolds are formed by freeze-casting a slurry that includes a biopolymer; during freeze-casting the biopolymer is excluded from forming ice crystals and deposits between the ice crystals to form the scaffold. In particular embodiments the biopolymer is selected from collagen, nanocellulose, xanthan gum, konjac glucomannan, alginate, agar, agarose, chitin, chitosan, chitosan-alginate polylactic acid (PLA), polygultamic acid (PGA,) polycaprolactone, or polydioxanone.

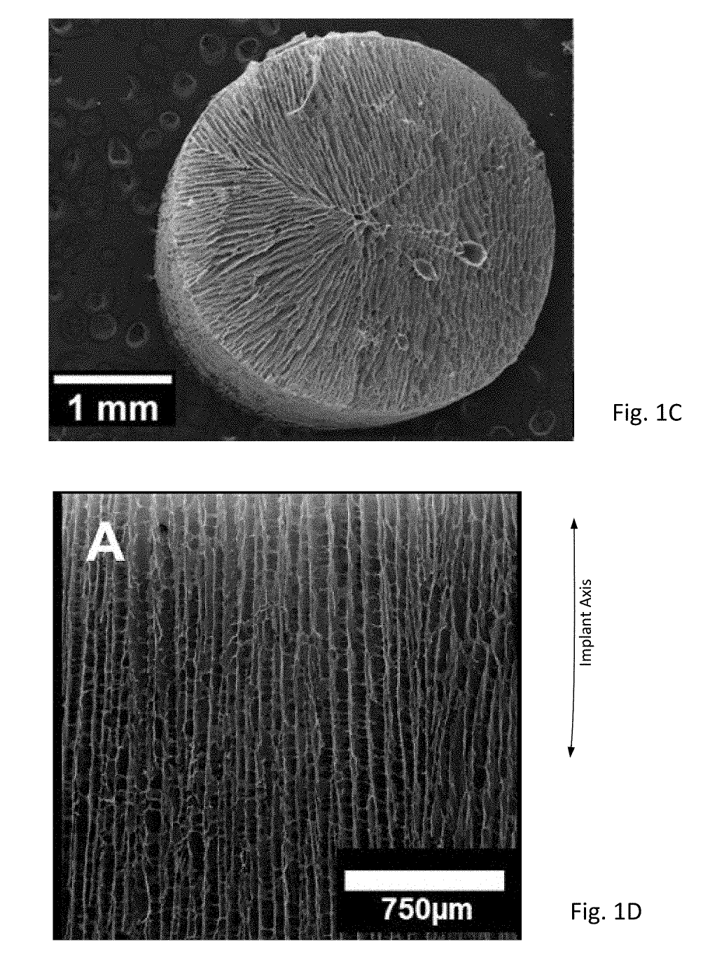

A prototype implantable device based on a Chitin (a polysaccharide) radially-freeze-cast scaffold is illustrated in cross section in FIG. 1A. A prototype implantable device based on a collagen-nanocellulose composite radially freeze-cast scaffold is illustrated in FIG. 1B. Another prototype implantable device formed of radially freeze-cast crosslinked bovine collagen illustrating radial porosity is illustrated in FIG. 1C. An implant 100 is shown implanted in a model of the intramural region of a fallopian tube 102 associated with a uterus 104 (only part of uterus 104 is shown) in FIG. 2. The uterus has a mouth or cervix 106 and a cavity 108 leading to a mouth 110 of fallopian tube 102. In particular embodiments, the freeze-cast scaffold has uncompressed diameter of between 2 and 10 millimeters, and length between 5 and 20 millimeters. In a particular embodiment diameter is four millimeters, while being approximately two centimeters long. In another particular embodiment, diameter is two millimeters while length is about two centimeters.

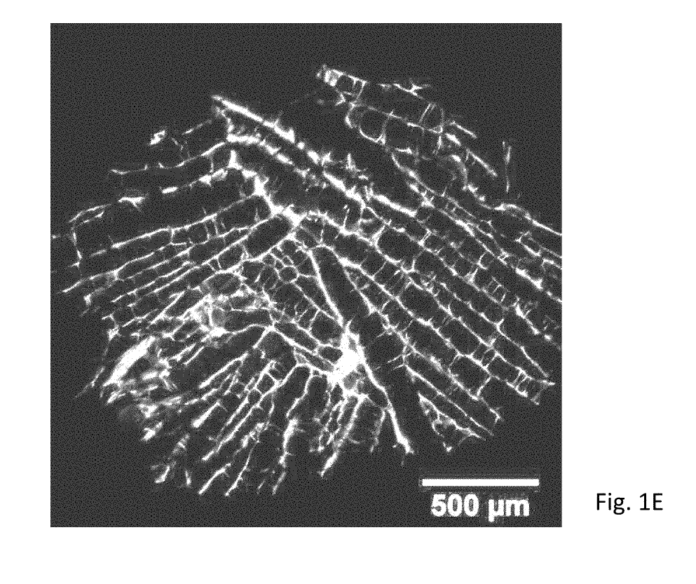

With reference to FIG. 3, the method of sterilization 200 begins by forming an implant by making a solution and/or slurry 202 according to a recipe, of which several examples are given below, then placing 204 the slurry in a mold having circular cross-section and freezing 206 the slurry under conditions where ice crystals grow radially (or otherwise) within or through the solution and/or slurry from the inner mold surface inwards towards a central axis of the mold. The frozen slurry, or green casting, is then removed from the mold and freeze-dried 208 without being allowed to melt. In some embodiments, the freeze-dried casting is chemically cross-linked 212, refrozen and re-freeze-dried 214. The freeze-dried casting is then inserted 216 into a smooth-walled plastic tubular insertion device having tip diameter under about two millimeters.

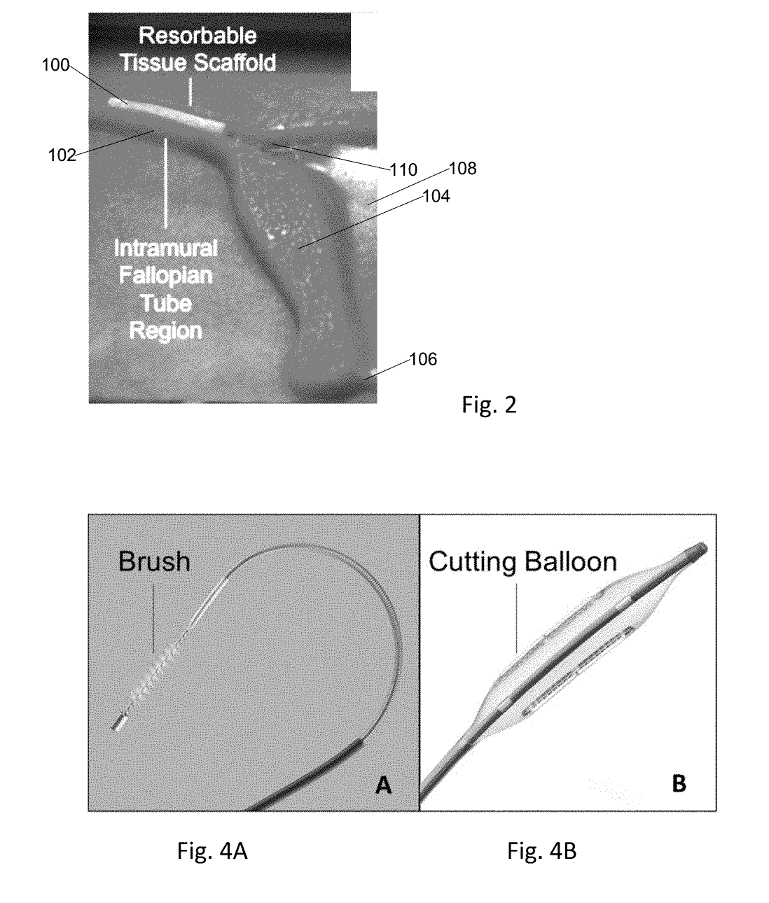

In embodiments, where mechanical abrasion of fallopian epithelium is performed to stimulate cell division and ingrowth of fibroblasts, an abrasion brush or balloon is used to abrade 218 the fallopian tube epithelium. In abrading the epithelium with an abrasion brush-catheter (FIG. 4A), the brush-catheter is inserted through the vagina, cervix, and uterus under hysteroscopic guidance into the fallopian tube, the brush is extended beyond its catheter, rotated to disrupt the fallopian tube epithelium, retracted into its catheter, and withdrawn. In abrading the epithelium with an abrasion balloon (FIG. 4B) mounted to the end of a catheter, the balloon is deflated, the catheter is routed into the fallopian tube under hysteroscopic guidance, the balloon is inflated, and withdrawn through a portion of the fallopian tube to scrape lining of the fallopian tube. The balloon is then deflated and the catheter is withdrawn.

Next, the tubular insertion device containing the implantable sterilization device at its tip is inserted 220 under hysteroscopic guidance into the Fallopian tube; the tube is then withdrawn 222 after or while expelling the implantable sterilization device from the tube using, for example, a pushrod within the lumen of the insertion device.

In devices relying on pre-insertion thermal injury, the tubular insertion device has a metallic circumferential electrode near its tip that is coupled through an insulated electrically conductive layer to an external electrocautery device. With this embodiment, the insertion device is positioned within the fallopian tube, the electrocautery device is activated briefly to damage epithelium of the fallopian tube, and the tubular insertion device is then withdrawn as the plunger pushes the implant out of the insertion device, leaving the implant within a portion of the fallopian tube where the epithelium was damaged by currents applied through the electrode to tissue.

In embodiments relying on post-insertion thermal injury to the fallopian tube lining to stimulate ingrowth of fibroblasts into the implantable sterilization device, using a sterilization device formed with a significant amount of magnetic nanoparticles, the device is exposed to an AC magnetic field 224 by applying an AC source to a coil positioned near the pelvic region of the patient. In particular embodiments, magnetic field concentrators may be used to enhance and direct the AC magnetic field produced by the coil to the pelvic region of the patient, where the fallopian tube is located. In a particular embodiment the AC source operates at a frequency between 100 kHz and 150 kHz.

The patient is then given a healing time to allow the patient's cells, such as fibroblasts, to grow into 226 the device while the biodegradable device itself degrades and is absorbed or expelled, leaving just the patient's cells blocking the Fallopian tube.

Steps 202-226 are repeated for the other Fallopian tube if one exists. Most species of placental mammals have two Fallopian tubes, although there are individual variations where only one patent Fallopian tube is present.

Since the implantable scaffolds described herein are porous, they may be saturated prior to insertion with a liquid or gel phase containing either or both of a chemical irritant or a drug. For example, in embodiments relying on chemical injury to the Fallopian tube lining to help induce infiltration of cells into the implant and scar formation, the implant may be saturated with a solution containing a chemical irritant. Once inserted into the Fallopian tube, the chemical irritant will leach out of the implant onto the Fallopian tube lining to cause chemical injury. Similarly, the liquid or gel phase may contain a drug that will leach out of the implant and may be absorbed through the Fallopian tube lining.

### Experimental Embodiments

In experimental embodiments, the slurry and scaffolds are prepared as follows:

Implants without Magnetic Nanoparticles

These recipes are those used to make particular experimental scaffolds that we have fabricated and tested in our lab. It is expected these scaffold recipes may be modified to make similar scaffolds containing magnetic nanoparticles by addition of magnetic nanoparticles to the suspension or slurry prior to freeze-casting.

1% Crosslinked Bovine Collagen-Nanocellulose (50:50) Blend

### Procedure:

1. Mass 1.00 grams of bovine Achilles tendon collagen
2. Add to 0.05 M Acetic acid to a total volume of 100 mL
3. Allow the heterogeneous solution to sit overnight at 4 degrees Celsius
4. Homogenize in an ice bath for 1-2 hours to create a uniform suspension/slurry
5. Store the 1% collagen slurry in the refrigerator and shear mix prior to use
6. Dilute 2.6% Nanoblend nanocellulose stock slurry to 1% by volume with distilled water
7. Store 1% nanocellulose slurry in the refrigerator and shear mix prior to use
8. Add by mass equal amounts of the 1% slurries (collagen and nanocellulose)
9. Shear mix the two-component mixture
10. Add the slurry to an aluminum mold via a needle/syringe and seal the bottom with a Teflon cap.
11. Freeze the slurry on the freezecaster coldfinger at a rate of −10° C./minute
12. Remove mold from coldfinger once the scaffold is frozen
13. Keep the mold in the −20° C. freezer for 15 minutes to increase the temperature
14. Punch scaffolds out of the mold
15. Lyophilize the frozen scaffolds for 24-36 hours

Chemical Crosslinking Procedure:
1. Stir scaffold in a 200-proof ethanol solution with 33 mM 1-Ethyl-3-(3-dimethylaminopropyl) carbodiimide and 6 mM N-hydroxysuccinimide for 6 hours at room temperature
2. Wash scaffolds three times for 1 hr., 12 hrs., and 1 hour, respectively; palpate scaffolds of fluid between washes
3. Submerse scaffolds in distilled water and flash freeze them in liquid nitrogen
4. Lyophilize scaffolds for 24-36 hours.

We have formed similar scaffolds with 1% and 3% collagen and expect implants can be made throughout this range, with 2% collagen scaffolds being stronger than those formed with 1% collagen.

1-3% Bovine Collagen with or without Magnetic Nanoparticles

1% to 3% collagen scaffolds (crosslinked and uncrosslinked), using bovine or jellyfish collagen, can also be made using the stated fabrication instructions without the addition of nanocellulose. One such procedure uses the following recipe to prepare slurry for freeze-casting:
1. A 1-3% w/v collagen slurry was prepared by adding 2 g of Type I fibrous bovine tendon collagen powder (Advanced Biomatrix Inc., San Diego, Calif.) to 0.05 M acetic acid,
2. refrigerating the mixture at 4 C for at least 12 hours, and
3. homogenizing (Fisher Scientific™ Homogenizer 152; Fisher Scientific International, Inc., Hampton, N.H.) thoroughly (at ˜¾ maximum rpm) on ice for between 1 and 2 hours.
4. For embodiments with magnetic nanoparticles, measure and add magnetic nanoparticles as freeze-dried powder, adding them slowly and both shear-mixing and vortex mixing prior to freeze-casting.
5. The slurry may be stored refrigerated prior to freeze casting.
6. Prior to freeze casting slurries were shear mixed for 2-3 minutes at 2100-2500 rpm (Speed Mixer™, DAC 150FVZ-K, FlackTek, Landrum, S.C.).
7. For radially-porous scaffolds, the slurry is inserted into an aluminum mold.
8. For longitudinally-porous scaffolds the slurry is inserted into a Teflon mold.
9. Freeze the slurry on the freezecaster coldfinger at a cooling rate between 10° C./minute-0.1° C./minute.
10. Remove mold from coldfinger once the scaffold is frozen
11. Keep the mold in the −20° C. freezer for 15 minutes to increase the temperature
12. Punch scaffolds out of the mold
13. Lyophilize the frozen scaffolds for 24-36 hours
14. Chemical crosslinking can be used with EDC-NHS or genipin

Based on theory, experimental immune-responses, structural stability, and potential for heating, bovine crosslinked collagen scaffolds having between 25 and 250 mg/cm of magnetic nanoparticles are expected to be manufacturable and useful for implantation into fallopian tubes for sterilization of female placental mammals, including humans and felines. We also expect these implants to be useful for implantation into fallopian tubes of canines. In these embodiments, the magnetic nanoparticles embedded in the scaffold serve as an agent for heating the scaffold and thermally damaging the fallopian tube lining.

We have found that cross-linked collagen, chitosan, and chitin scaffolds exhibit shape memory. Shape-memory allows a scaffold that has been formed at a first diameter, then compressed to a second diameter for insertion into the insertion tool, to re-expand to near the first diameter after insertion into the fallopian tube to fill voids and more effectively block the tube than if the diameter stayed constant.

0.6% Chitin

Procedure:
1. Store 0.6% chitin stock slurry in the refrigerator and shear mix prior to use
2. Add the slurry to the aluminum mold via a needle/syringe and seal the bottom with a Teflon cap.
3. Freeze the slurry on the freezecaster coldfinger at a rate of −10° C./minute
4. Remove mold from coldfinger once the scaffold is frozen
5. Keep the mold in the −20° C. freezer for 15 minutes to increase the temperature
6. Punch scaffolds out of the mold
7. Lyophilize scaffolds for 24-36 hours

Based on theory, experimental immune-response, structural stability, and potential for heating, chitin scaffolds having between 25 and 250 mg/cm of magnetic nanoparticles are expected to be useful for implantation into fallopian tubes for sterilization of female placental mammals, including humans and felines. We also expect these implants to be useful for implantation into fallopian tubes of canines.

### 2-8% Chitosan Scaffolds

Procedure:
1. Mass 2-8 grams of Chitosan powder and add to 0.05-0.10 M Acetic acid to a total volume of 100 mL
2. Allow the mixture to dissolve into a homogenous solution with a roller mixer for 1-5 days
3. Add the slurry to an aluminum mold via a needle/syringe and seal the bottom with a Teflon cap for radial freeze casting
4. Add the slurry to a Teflon mold via a needle/syringe and seal the bottom with a copper cap for longitudinal freeze casting
5. Freeze the slurry on the freezecaster coldfinger at a cooling rate between 10° C./minute-0.1° C./minute.
6. Remove mold from coldfinger once the scaffold is frozen
7. Keep the mold in the −20° C. freezer for 15 minutes to increase the temperature
8. Punch scaffolds out of the mold
9 Lyophilize the frozen scaffolds for 24-36 hours
10 Stabilization procedure:
11 Stir scaffold in 0.4 v/v % sodium hydroxide solution in 95% ethanol for between 0.5-24 hours.
12 Wash scaffolds three times for 1 hr., 12 hrs., and 1 hour, respectively in distilled water
13 Submerse scaffolds in distilled water and flash freeze them in liquid nitrogen
14 Lyophilize scaffolds for 24-36 hours.

### Magnetic Nanoparticle (MNP)-Containing Implants.

Nanocellulose+MNPs with 25 mg/ml Iron Content:

Materials:
2101 mg UMaine stock nanocellulose (2.1% solid)
2138 mg water (to dilute the stock to 1% solid)
200.3 mg MNPs (100 nm BNF Dextran)

Procedure:
1. Mass container (small container w/ screw-on lid)
2. Add nanocellulose and water to container
3. Shear mix to combine nanocellulose and water
4. Add MNPs to container
5. Shear mix until all ingredients combined
6. Mass aluminum mold with Teflon cap
7. Use syringe to fill mold; record amount of material in mold
8. Freeze on coldfinger at −10° C./minute
9. Remove mold from coldfinger once scaffold frozen
10. Keeping mold in the −80° C. freezer, punch scaffold out of mold
11. Lyophilize scaffold for 48 hours

Xanthan-Konjac Glucomannan Gum (Gelled)+MNPs with 25 mg/ml Iron Content:

Materials:
4332 mg XK gum (090316-02, 3:2 X:K, pH 5, 1%, contains azide)

The term XK gum is used herein to indicate a xanthan gum and konjac glucomannan combination. The stock powders were dissolved in a xanthan:konjac ratio of 3:2 in double distilled water and adjusted to a pH of 5 (with citric acid and sodium hydroxide) to achieve 1% m/v slurry.
207.6 mg MNPs (100 nm BNF Dextran)

Procedure:
1. Mass container (small container w/ screw-on lid)
2. Add XK gum to container
3. Add MNPs to container
4. Shear mix until ingredients combined
5. Gel gum: place container in plastic bag in water bath. Once water reaches 90° C., leave gum in water bath for two hours
6. Mass aluminum mold with Teflon cap
7. Use syringe to fill mold;
8. Freeze on coldfinger at −10° C./minute
9. Remove mold from coldfinger once scaffold frozen
10. Keeping mold in the −80° C. freezer, punch scaffold out of mold
11. Lyophilize scaffold for 48 hours

Xanthan-Konjac Glucomannan Gum (Ungelled)+MNPs with 25 mg/ml Iron Content

Materials:
3915 mg XK gum (090316-02, 3:2 X:K, pH 5, 1%, contains azide)
181 mg MNPs (100 nm BNF Dextran)

Procedure:
1. Mass container (small container w/ screw-on lid)
2. Add XK gum to container
3. Add MNPs to container
4. Shear mix until ingredients combined
5. Mass aluminum mold with Teflon cap
6. Use syringe to fill mold; record amount of material in mold
7. Freeze on coldfinger at a rate of −10° C./minute
8. Remove mold from coldfinger once scaffold frozen
9. Keeping mold in the −80° C. freezer, punch scaffold out of mold
10. Lyophilize scaffold for 48 hours

Nanocellulose+MNPs with 250 mg/ml Iron Content:

Materials:
756 mg UMaine stock nanocellulose—1% solid
456 mg MNPs (100 nm BNF Dextran)

Procedure:
1. Mass eppendorf
2. Add nanocellulose to eppendorf
3. Add MNPs slowly and shear mix and vortex to combine between additions
4. Mass aluminum mold with Teflon cap
5. Use syringe+18.5 gauge needle to fill mold; record amount of material in mold
6. Freeze on coldfinger at a rate of −10° C./minute
7. Remove mold from coldfinger once scaffold frozen
8. Keeping mold in the −80° C. freezer, punch scaffold out of mold
9. Lyophilize scaffold for 48 hours

Xanthan-Konjac Glucomannan Gum (Ungelled)+MNPs at 250 mg/ml Iron Content:

Materials:
743 mg XK gum (090316-02, 3:2 X:K, pH 5, 1%, contains azide)
436 mg MNPs (100 nm BNF Dextran)

Procedure:
1. Mass eppendorf
2. Add XK gum to eppendorf
3. Add MNPs slowly and shear mix and vortex to combine between additions
4. Mass aluminum mold with Teflon cap
5. Use syringe+18.5 gauge needle to fill mold; record amount of material in mold
6. Freeze on coldfinger at −10° C./minutes
7. Remove mold from coldfinger once scaffold frozen
8. Keeping mold in the −80° C. freezer, punch scaffold out of mold
9. Lyophilize scaffold for 48 hours

Xanthan-Konjac Glucomannan Gum with 540 mg/ml Iron Content

Materials:
274 mg XK gum
518 mg MNPs or 288 mg Fe (100 nm BNF Dextran)

Procedure:
1. Mass eppendorf
2. Add XK gum to eppendorf (ungelled)
3. Add MNPs slowly and shear mix and vortex to combine between additions
a. 109.5 mg, 91.02 mg, 262.15 mg, 7.65 mg, 17.8 mg, 30 mg for a the total of 518 mg of MNPs
4. According to traditional density based calculations—final iron concentration: ˜540 Fe mg/mL
5. Mass Ratio of 1% XK gum: Fe 274 mg:288 mg=0.951:1

### Experimental Results

Scaffolds formed from XK (Xantan-Konjac blend) gum and bovine collagen are mechanically stronger if the material is crosslinked as a gel prior to freeze-casting; this added mechanical strength is helpful in implantation. Further, we found scaffolds with 250 Fe mg/mL can be formed despite the high concentration of MNPs giving a viscous freezing slurry with low amounts of water; the freeze casting process gave a minimally porous material that maintained structural integrity; we believe scaffolds with concentrations of between 25 and 250 Fe/ml can be formed. With XK-gum we found gelation could unlock a host of structurally and morphologically attractive materials.

We have implanted into mice scaffolds made from (A) uncrosslinked bovine collagen; (B) crosslinked bovine collagen; (C) crosslinked bovine collagen-nanocellulose blend; (D) nanocellulose blend; (E) crosslinked jellyfish collagen; (F) chitin; (G) xanthan-konjac glucomannan (XK); and (H) control devices made according to the above recipes subcutaneously into mice, and have observed that the implants become surrounded by and infiltrated with fibroblasts within 30 days with XK gel provoking the most intense immune response and chitin the least. We have observed that nanocellulose-bovine collagen scaffolds become infiltrated with macrophages and neutrophils.

We have also demonstrated thermal occlusion of Fallopian tubes in felis cattus (domestic cats), by applying microwave thermal energy directly to the Fallopian tube exterior.

We have demonstrated heating of scaffolds incorporating magnetic nanoparticles in an AC magnetic field at rates of 0.625 watts per cubic centimeter. It is expected that, by using different AC magnetic field strengths or frequencies, and possibly alternative sizes and types of magnetic nanoparticles, heating of the scaffolds in an AC magnetic field may be increased significantly to about 10 watts per cubic centimeter.

In embodiments, fibroblast infiltration and closure of the Fallopian tube lumen is complete in about 30 days.

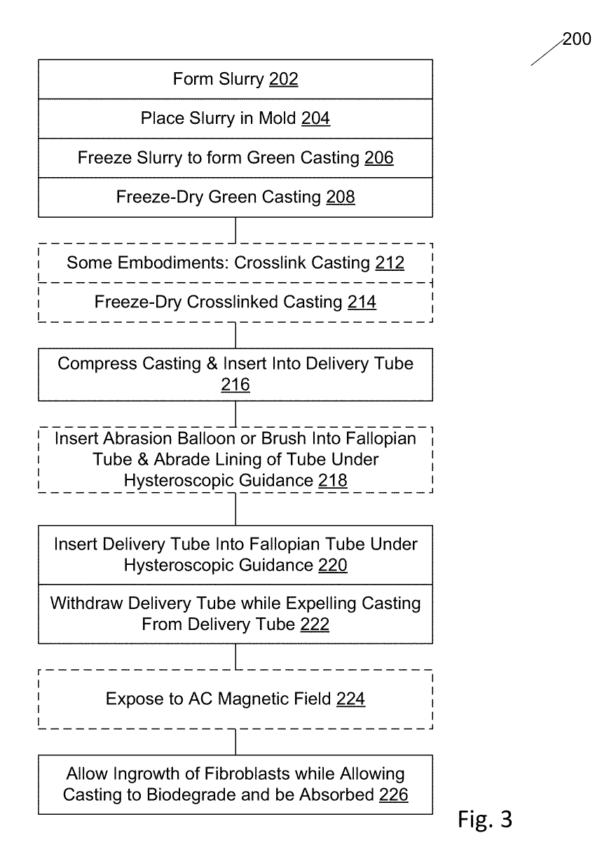

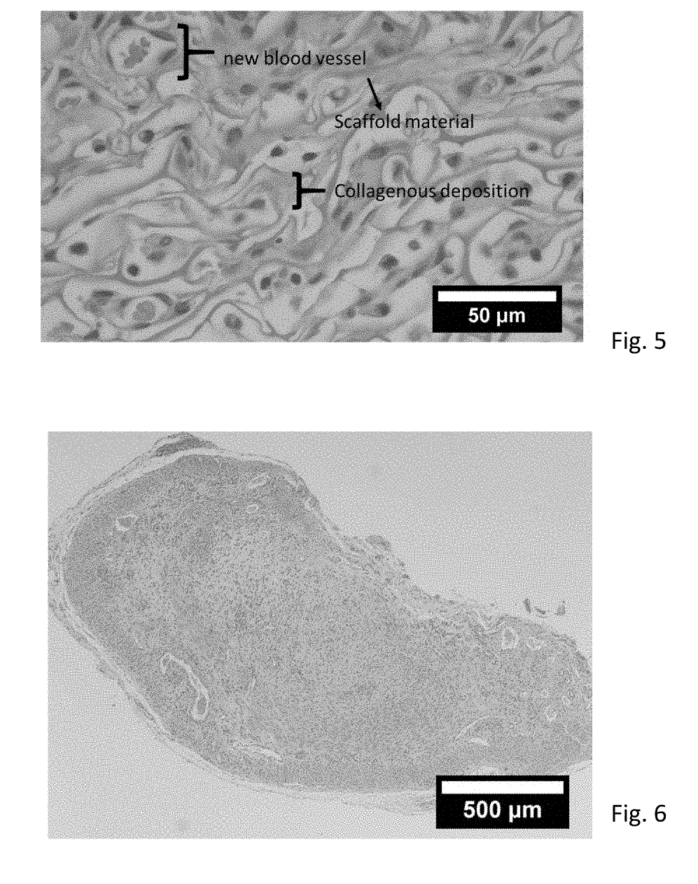

We have also formed 2 mm diameter scaffolds by punching out longitudinally-porous 2% bovine collagen scaffolds with a biopsy punch, then sterilized them in ethylene oxide. The scaffolds were then implanted into rat uterine horn following mechanical disruption of the lining. FIG. 5 illustrates cellular infiltration into the scaffold 6 days after implantation, and FIG. 6 illustrates obliteration of the uterine horn lumen at 30 days after implantation, both micrographs were taken of tissue fixed in formalin and stained with hematoxylin and eosin. While we were successful at achieving cellular infiltration and lumen obliteration with longitudinally porous scaffolds, we expect more rapid cellular infiltration with radially-porous scaffolds.

It is believed that crosslinked collagen or chitin radially freeze-cast scaffolds containing at least 25 mg/cc of magnetic nanoparticles and uncompressed diameter of between one and four millimeters, and in an embodiment two millimeters, offer a good chance of success as a bioabsorbable, implantable, sterilization device in placental mammals including humans, felines, and canines.

### Alternative Scaffold Materials

While experimental devices have been made from bovine collagen-nanocellulose mix, XK gum, chitin, bovine collagen alone, and nanocellulose alone, all with and without magnetic nanoparticles, it is expected that devices may be made using other biopolymers such as alginate, agar, agarose, chitin, chitosan, chitosan-alginate polylactic acid (PLA), polygultamic acid (PGA), polycaprolactone, or polydioxanone. It is anticipated that devices may contain magnetic nanoparticles and/or drugs intended to regulate or enhance fibroblast ingrowth.

### Chemical Epithelial Disruption

In an alternative embodiment, instead of or in addition to mechanical abrasion of fallopian tube lining or heating to damage the fallopian tube lining, a chemical irritant such as hydrogen peroxide is applied to pores of the implant. After implantation of the implant, the chemical irritant serves to damage the epithelium, thereby encouraging rapid cell division. With this embodiment, the chemical irritant should be selected such that it will be metabolized, expelled, or diluted sufficiently rapidly that cells, such as fibroblasts, can infiltrate the implant unimpeded by the irritant.

In an alternative embodiment, the implant contains both a chemical irritant and magnetic nanoparticles adapted to heat the implant when exposed to an AC magnetic field. This embodiment is adapted to de-epithelialize the fallopian tube lining by a combination of heating and chemical irritation.

### Drug-Doped Scaffolds

Drugs that may be incorporated into the device scaffold by adding to the slurry either the drug itself, or microbeads or microcapsules containing the drug prior to freeze-casting the device. In alternative embodiments, drugs are applied as a coating to the scaffold, or infiltration of a liquid bearing the drug into the scaffold.

Drugs anticipated as ingredients of the slurry include copper or copper compounds, hormonal contraceptives, growth and/or other factors such as a chemical de-epithelialization agent that would serve to stimulate fibroblast ingrowth without requiring mechanical or thermal de-epithelialization.

### Combinations

It is anticipated that the materials, tools, and methods discussed herein may be combined in a variety of ways. Among these are:

An implantable device designated A configured for insertion into a fallopian tube and adapted to obstruct the fallopian tube, including a radially-porous polymeric scaffold including at least one material selected from the group consisting of chitin, collagen, nanocellulose, xanthan gum, konjac glucomannan, chitin, alginate, agar, agarose, chitosan, chitosan-alginate polylactic acid (PLA), polygultamic acid (PGA,) polycaprolactone, and polydioxanone. The device is compressed and configured within a first end of a tube having smooth interior walls, the tube being flexible and adapted for insertion through a cervix into the fallopian tube and the tube is equipped with a plunger adapted to expel the device from the tube.

A device designated AA including the device designated A wherein the radially-porous polymeric scaffold is biodegradable.

A device designated AB including the device designated A or AA wherein the radially-porous polymeric scaffold is formed by freeze-casting a gel selected from the group consisting of a blend of xanthan gum and konjac glucomannan, and crosslinked collagen.

A device designated AC including the device designated A, AA or AB wherein the radially-porous polymeric scaffold includes collagen and nanocellulose.

A device designated AD including the device designated A, AA, AB, or AC wherein the radially-porous polymeric scaffold includes at least 25 milligrams of iron per milliliter of scaffold prior to compression of the device.

A device designated AE including the device designated A, AA, AB, AC, or AD wherein the radially-porous polymeric scaffold comprises copper.

A device designated AF including the device designated A, AD or AE wherein the device comprises magnetic nanoparticles in a range between 25 and 500 250 milligrams of iron per cubic centimeter of uncompressed device.

A device designated AG including the device designated A, AA, AB, AC, AD, AE, or AF wherein the radially-porous polymeric scaffold has diameter of approximately 4 millimeters prior to compression and insertion into the tube.

A method designated B of sterilization of a female placental mammal includes: using a tubular insertion device containing a radially freeze-cast and freeze-dried implant formed of a polymer selected from the group consisting of chitin, collagen, nanocellulose, xanthan gum, konjac gum, chitin, alginate, agar, agarose, chitosan, chitosan-alginate polylactic acid (PLA), polygultamic acid (PGA,) polycaprolactone, and polydioxanone; and under hysteroscopic guidance expelling the implant from the tubular insertion device into a Fallopian tube of the female mammal; disrupting epithelium of the Fallopian tube; and allowing ingrowth of cells into the implant.

A method designated BA including the method designated B wherein the implant is at least partially biodegradable.

A method designated BB including the method designated B or BA wherein the radially-porous polymeric scaffold comprises gel selected from the group consisting of a blend of xanthan gum and konjac glucomannan, and crosslinked collagen and at least 25 milligrams of iron per milliliter of scaffold prior to compression of the device.

A method designated BC including the method designated B, BA, or BB wherein the step of disrupting epithelium of the Fallopian tube is performed by exposing the device is exposed to an AC magnetic field after expelling the implant from the tubular insertion device into the Fallopian tube.

A method designated BD including the method designated B, BA, BB, or BC wherein the radially-porous polymeric scaffold has diameter of approximately 4 millimeters prior to compression and insertion into the tube.

A method designated BD including the method designated B, BA, BB, or BC wherein the radially-porous polymeric scaffold comprises collagen and nanocellulose.

A method designated BE including the method designated B, BA, BB, BC, or BD wherein the step of disrupting epithelium of the Fallopian tube is performed prior to expelling the implant from the tubular insertion device into the Fallopian tube and comprises mechanical abrasion performed with a device selected from the group consisting of an abrasion brush and an abrasion balloon.

A method designated BF including the method designated B, BA, BB, BC or BD wherein the step of disrupting epithelium of the Fallopian tube is performed by exposing the device is exposed to an AC magnetic field after expelling the implant from the tubular insertion device into the Fallopian tube, and wherein the implant comprises magnetic nanoparticles.

A method designated BG including the method designated B, BA, BB, BC or BD wherein the step of disrupting epithelium of the Fallopian tube is performed by coupling an electrocautery device through an insulated conductor of the tubular insertion device to an electrode near a tip of the tubular insertion device prior to expelling the implant from the tubular insertion device into the Fallopian tube.

A method designated BH including the method designated B, BA, BB, BC, BD, BE, BF, or BG wherein the female placental mammal is human.

A method designated BJ including the method designated B, BA, BB, BC, BD, BE, BF, or BG wherein the female placental mammal is feline.

Additional potential combinations include:

A device designated C including the implantable device designated A wherein the device comprises growth factors.

A device designated D including the implantable device designated A wherein the device comprises contraceptive hormones.

A scaffold designated E including a radially-porous scaffold, wherein the polymer comprises at least one selected from the group consisting of collagen, cellulose, chitin, chitosan, alginate, agar, agarose, gelatin, soy protein, hyaluronic acid, elastin, silk, xanthan gum, konjac glucomannan, polylactic acid (PLA), polygultamic acid (PGA), polycaprolactone, or polydioxanone, or any combination thereof.

A delivery device designated F for the placement of the scaffold designated E into a fallopian tube configured to hold the scaffold designated E within the tip of a tube having smooth interior walls, the tube being flexible and adapted for insertion through the vagina, cervix, and into the fallopian tube. The delivery device equipped with a plunger adapted to expel the device from the tube.

A method designated G of sterilization of a female placental mammal comprising: using the scaffold designated E to place the scaffold under hysteroscopic guidance.

The method designated G, placing the implant without disrupting epithelium of the Fallopian tube.

A method designated J including the method designated G, placing the implant after disrupting the epithelium of the Fallopian tube.

A method designated K including the method designated G, placing the implant, disrupting the epithelium of the Fallopian tube after implant placement.

A method designated L including the method designated J or K, disrupting the epithelium of the Fallopian tube mechanically.

The method designated J or K, disrupting the epithelium of the Fallopian tube chemically.

A method designated M including the method designated J or K, disrupting the epithelium of the Fallopian tube by heating.

The method designated L wherein the step of disrupting epithelium of the Fallopian tube is performed prior to expelling the implant from the tubular insertion device into the Fallopian tube and comprises mechanical abrasion performed with a device selected from the group consisting of an abrasion brush and an abrasion balloon.

The method designated M wherein the step of disrupting epithelium of the Fallopian tube is performed by exposing the device is exposed to an AC magnetic field after expelling the implant from the tubular insertion device into the Fallopian tube, and wherein the implant comprises magnetic nanoparticles.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An implantable device configured for insertion into a fallopian tube and adapted to obstruct the fallopian tube, the device comprising a porous polymeric scaffold comprising a biopolymer;
wherein the device is compressed and configured within a first end of a tube having smooth interior walls, the tube being flexible and adapted for insertion through a cervix into the fallopian tube and the tube is equipped with a plunger adapted to expel the device from the tube.

2. The device of claim 1, wherein the porous polymeric scaffold is biodegradable and the biopolymer comprises at least one material selected from the group consisting of collagen, nanocellulose, xanthan gum, konjac glucomannan, chitin, alginate, agar, agarose, chitosan, chitosan-alginate polylactic acid (PLA), polygultamic acid (PGA,) polycaprolactone, and polydioxanone.

3. The device of claim 2, wherein the radially-porous polymeric scaffold comprises gel selected from the group consisting of a blend of xanthan gum and konjac glucomannan, and crosslinked collagen, and comprises at least 25 milligrams of iron per milliliter of scaffold prior to compression of the device.

4. The device of claim 3, wherein the porous polymeric scaffold has diameter between one and four millimeters prior to compression and insertion into the tube.

5. The device of claim 4 wherein the porous polymeric scaffold has diameter approximately two millimeters.

6. The device of claim 2, wherein the porous polymeric scaffold comprises collagen.

7. The device of claim 1, wherein the porous polymeric scaffold comprises copper.

8. The device of claim 1, wherein the porous polymeric scaffold comprises magnetic nanoparticles in a range between 25 and 250 milligrams of iron per cubic centimeter of uncompressed radially-porous polymeric scaffold.

9. A method of sterilization of a female placental mammal comprising:
using a tubular insertion device containing a freeze-cast and freeze-dried implant formed of a biopolymer selected from the group consisting of chitin, collagen, nanocellulose, xanthan gum, konjac gum, chitin, alginate, agar, agarose, chitosan, chitosan-alginate polylactic acid (PLA), polygultamic acid (PGA,) polycaprolactone, and polydioxanone;
and under hysteroscopic guidance expelling the implant from the tubular insertion device into a Fallopian tube of the female mammal;
disrupting epithelium of the Fallopian tube; and
allowing ingrowth of cells into the implant.

10. The method of claim 8, wherein the implant is at least partially biodegradable.

11. The method of claim 9, wherein the radially-porous polymeric scaffold comprises gel selected from the group consisting of a blend of xanthan gum and konjac glucomannan, and crosslinked collagen and at least 25 milligrams of iron per milliliter of scaffold prior to compression of the device.

12. The method of claim 10, wherein the step of disrupting epithelium of the Fallopian tube is performed by exposing the device is exposed to an AC magnetic field after expelling the implant from the tubular insertion device into the Fallopian tube.

13. The device of claim 9, wherein the radially-porous polymeric scaffold has diameter of between approximately one and four millimeters prior to compression and insertion into the tube.

14. The device of claim 9, wherein the radially-porous polymeric scaffold comprises collagen and nanocellulose.

15. The method of claim 9, wherein the step of disrupting epithelium of the Fallopian tube is performed prior to expelling the implant from the tubular insertion device into the Fallopian tube and comprises mechanical abrasion performed with a device selected from the group consisting of an abrasion brush and an abrasion balloon.

16. The method of claim 9, wherein the step of disrupting epithelium of the Fallopian tube is performed by exposing the device is exposed to an AC magnetic field after expelling the implant from the tubular insertion device into the Fallopian tube, and
wherein the implant comprises magnetic nanoparticles.

17. The method of claim 9, wherein the step of disrupting epithelium of the Fallopian tube is performed by coupling an electrocautery device through an insulated conductor of the tubular insertion device to an electrode near a tip of the tubular insertion device prior to expelling the implant from the tubular insertion device into the Fallopian tube.

18. The method of claim 9, wherein the female placental mammal is human.

19. The method of claim 9, wherein the female placental mammal is feline.

20. The method of claim 9, wherein the female placental mammal is canine.

21. The method of claim 9, wherein, prior to expelling the implant into the Fallopian tube the implant is saturated with a solution containing a chemical irritant, and wherein the step of disrupting epithelium of the Fallopian tube is performed by the chemical irritant.

22. The device of claim 4 wherein the scaffold has diameter of approximately two millimeters prior to compression into the insertion device.

23. An implantable device configured for insertion into a fallopian tube and adapted to obstruct the fallopian tube, the device comprising a porous polymeric scaffold comprising at least one material selected from the group of biopolymers or biodegradable polymers; wherein the device is compressible and configurable within a first end of a tube adapted for insertion through a cervix into the fallopian tube, and the tube is equipped with an expulsion device configured to expel the device from the tube.

24. The implantable device of claim 23, further comprising a liquid or gel phase disposed within the implantable device, the liquid phase further comprising an agent selected from the group of chemical irritants and drugs.

25. The implantable device of claim 24 wherein the agent is a chemical irritant adapted to at least partially de-epithelialize the fallopian tube.

26. The device of claim 25 further comprising magnetic nanoparticles adapted to heat the scaffold when exposed to an AC magnetic field.

27. The device of claim 23, wherein the longitudinally, or radially-porous, or mixed-type porous polymeric scaffold comprises an agent for thermal de-epithelialization of the fallopian tubes.

28. The device of claim 23, wherein the scaffold is formed by freeze-casting.
